(19) United States
(12) Patent Application Publication
(10) Pub. No.: US 2020/0352745 A1
(43) Pub. Date: Nov. 12, 2020

(54) POWER SUPPLY FOR A PROSTHESIS

(71) Applicant: Stefan SCHULZ, Karlsruhe (DE)

(72) Inventor: Stefan SCHULZ, Karlsruhe (DE)

(21) Appl. No.: 16/765,251

(22) PCT Filed: Nov. 22, 2018
(86) PCT No.: PCT/EP2018/000525

§ 371 Date: May 19, 2020

(30) Foreign Application Priority Data
Nov. 23, 2017 (DE) 10 2017 010 839.0

(51) Int. Cl. A61F 2/70; H02J 7/00; A61F 2/54

(52) U.S. Cl. CPC A61F 2/70; A61F 2002/701; A61F 2/54; H02J 7/0024

(57) ABSTRACT

Energy supply system 3 for a prostheses 1, comprising at least a first 12 and a second 16 energy storage means, wherein in a charging mode for charging at least the first energy storage means 12, the first energy storage means 12 is electrically connected to the second energy storage means 16 in a first manner and in a working mode for delivering electrical energy to a consumer the first energy storage means 12 is electrically connected to the second energy storage means 16 in a second manner, such that the first connecting manner is different from the second connecting manner.

This application is a National Stage completion of PCT/EP2018/000525 filed Nov. 22, 2018, which claims priority from German patent application serial no. 10 2017 010 839.0 filed Nov. 23, 2017.

### FIELD OF THE INVENTION

The invention relates to an energy supply system for a prosthesis, according to the preamble of the first principal claim, and to a prosthesis that comprises such an energy supply system and to a method for charging an energy storage means.

### BACKGROUND OF THE INVENTION

Prostheses can be used to support or replace limbs, in particular human limbs. For that purpose prostheses, such as exoprostheses and/or orthoses, can be designed to be inherently movable. In modern prostheses electric actuators may be present, for example in order to move parts of the prosthesis or to influence the properties of passive elements such as damping elements. The electrical energy required by those actuators can be stored in one or more energy storage means. To be able to supply the necessary energy, the energy storage means are supplied or charged with energy from a further source of energy. For this the energy storage means can be connected to a charging device, which is located within or outside the prosthesis or both within and outside the prosthesis.

### SUMMARY OF THE INVENTION

The objective of the invention is to provide an energy supply system for a prosthesis, a prosthesis and a method for charging an energy storage means of a prosthesis, which develop further the prior art.

A further objective can be to provide an energy supply system for a prosthesis, which can be charged by means of an energy source with a low charging voltage, as well as a corresponding prosthesis and a method for charging the energy storage means.

This objective is achieved by an energy supply system, a prosthesis and a method for charging an energy storage means according to the independent claim(s).

Particular embodiments are described in the subordinate claims.

In particular the objective is achieved by an energy supply system for a prosthesis which comprises at least a first and a second energy storage means for the storage of electrical energy, such that in one charging mode for charging the at least one energy storage means the first energy storage means is electrically connected to the second energy storage means in a first manner and in a working mode, in order to deliver the electrical energy from the first energy storage means to a consumer, the first energy storage means is electrically connected to the second energy storage means in a second manner, such that the first switching mode is different from the second switching mode.

Such a prosthesis can be an exoprosthesis or an orthosis. Preferably, such a prosthesis is an arm, a hand, a partial hand or a finger prosthesis. Thus, it can be a prosthesis with actively movable fingers and/or a movable wrist. In particular the consumer can be arranged in the metacarpus or in the fingers of the prosthesis.

The consumers can in this case be electric motors, but also control and regulation units and/or display units such as screens and LEDs, etc.

One or more fingers can be arranged and able to pivot on a basic hand body and can be moved about their pivot axis by the electric motor. In such a case one or more motors can be associated with each finger. One motor can also be connected to more than one finger.

The one or more motors can be arranged in the fingers and can move the fingers relative to the basic body of the hand via a transmission, such as a worm gear.

The fingers can have one or more joints, with parallel or offset rotation axes.

The energy supply system can consist of at least a first and a second energy storage means, which are preferably arranged in or on the prosthesis. In fact, it is even possible for more than two energy storage means to be connected to the consumers. In such a case one of the two energy storage means, more than one energy storage means or all the storage means can consist of a single energy accumulator unit or several energy accumulator units. Thus, a number of energy accumulator units can be connected electrically to one another in a fixed or variable manner to form an energy storage means.

The first and second energy storage means can be rechargeable electrical energy accumulators.

It has been recognized that the requirements demanded from the energy storage means during the charging mode differ from the requirements demanded from the energy storage means during the working mode. To be able to fulfill these different requirements, it can for example be expedient to change the switching mode between the energy storage means when changing between the charging mode and the working mode.

In this context a switching mode is understood to mean whether the electric outputs or inputs of one of the energy storage means are electrically connected to the electric outputs and/or inputs of the other energy storage means and whether, for example, the electrically negative output of the first energy storage means is electrically connected to the electrically positive output of the second energy storage means or to the electrically negative output of the second energy storage means.

The electrically negative output of an energy storage means can be the electrically negative input of the energy storage means and/or also the electrically negative pole of the energy storage means.

The electrically positive output of an energy storage means can be the electrically positive input of the energy storage means and/or the electrically positive pole of the energy storage means.

In the working mode, the first energy storage means can be connected in series with the second energy storage means. Thus, the electrically negative input of the first energy storage means can be electrically connected to the electrically positive input of the second energy storage means and the electrically positive output of the first energy storage means can be connected to an electrically positive input of a consumer. The electrically negative output of the second energy storage means can in this case be connected to the electrically negative input of the consumer. Thus, for example, during the working mode the energy storage means can be charged with a lower voltage and yet deliver a higher voltage to the one or more consumers. By virtue of this higher voltage there is no need for higher currents even when higher powers are desired, and for that reason smaller conductor and conducting path cross-sections for cables and smaller electronic components can be used.

In the charging mode the first energy storage means can be connected in parallel with the second energy storage means. Thus, lower voltages can be used for charging the energy storage means and, for example, conventional charging units can be used. Thanks to this parallel connection of at least the first and second energy storage means, it is also possible to use only one charging device for both of the energy storage means and thus to construct the prosthesis with only a few individual components.

In this context the charging device can be a device suitable for connecting an external current source electrically to at least one energy storage means. In particular, a charging device can deliver electrical energy to the energy storage means during the charging process. The charging device can comprise one or more switching devices, such as mechanical or electrical switches, relays, etc., in order to be able to change between the charging mode and a working mode. The charging device can also comprise a controller that can limit and/or regulate or control the charging current and/or the charging voltage.

The charging device can be connected to an external current source such as a mains network or an external energy storage device such as a charging battery or charging accumulator, or a solar module. A connecting conductor can also be connected between the charging device and the external charging device, which conductor for example brings the voltage of the external current source, for example by means of a transformer, down to the input voltage of the charging device. For example, the connecting conductor can bring the voltage of 220 V, 230 V or 110V corresponding to the mains network down to 4.4 V to 5.25 V.

In the charging mode the first energy storage means and the second energy storage means can also be separated from one another, in particular electrically. Thus for example, the first energy storage means and the second energy storage means can each be connected to a respective charging device for charging the energy storage means concerned. For this, for example, two charging devices can be used, the first charging device connected to the first energy storage means and the second charging device to the second energy storage means. The same charging device can also be used for both of the energy storage means, but with the charging device connected to the first and to the second energy storage means with a time offset or at separate times. In that way an additional charging device can be avoided.

Preferably, the energy storage means is, in particular, a lithium-ion accumulator. Other accumulators or structural forms of the lithium-ion accumulator are also conceivable, for example lithium polymer accumulators.

The charging device can comprise a connection means, such as a plug according to the USB (Universal Serial Bus) standard, and in particular a charging device can be provided which for example supplies to the connection means, as its input voltage, a voltage between 4.4 and 5.25 volts and thus corresponds to a USB standard such as USB Standard 2.0, USB 3.0, Micro USB and USB-C Standard. The charging device can also comprise a connection means which is compatible with the standard USB charging plug and thus delivers a voltage of 4.75 to 5.25 volts as the input voltage range.

The connection means can be suitable for connecting the charging device to one or more external current sources. The charging device can also comprise a device such as a coil, which enables inductive charging of the energy storage means.

In particular the energy supply system can comprise a plurality of energy storage means, such as at least the first, the second and a third energy storage means, wherein in the working mode at least the majority of the energy storage means to be charged are connected in series in each case to at least one of the energy storage means and in the charging mode the majority are in particular connected to one another in a manner which is different from the manner of their connection in the working mode.

It is also possible for the energy supply system to comprise a plurality of accumulators, wherein in a working mode one group of the accumulators are connected in series and in a charging mode they are connected in a manner which differs from the series connection. Preferably, in the charging mode the group can be connected in parallel or electrically separated from one another.

The energy supply system can comprise a manual switch for switching between the charging mode and the working mode. Such a switch can allow the user to switch between the working mode and the charging mode. It is also possible to provide several switches, which allow switching for example from an accumulator in the charging mode while another accumulator together with a third accumulator supply the consumer with energy, for example being connected in series.

The connection mode of the accumulators can be changed by means of electric, magnetic or electronic switches or relays.

The energy supply system can also comprise a detection device in order to detect whether a charging means such as a power supply unit should be connected to the energy storage means. Thus, the detection device can detect whether a charging means is connected to an input such as a connection means which is connected to the accumulator to be charged during the charging process. If the charging means is detected at the input by the detection device, the charging process can be initiated for example by connecting the accumulators to one another in the appropriate manner.

For example, the detection device can determine whether a plug is connected to a charging cable, and if necessary also determine whether a voltage is applied to the charging cable. If so the charging process can be initiated, i.e. for example the accumulators can be connected in parallel or separated.

The detection device can also comprise switches and/or relays, which separate and/or change the connections between the accumulators.

The first and second energy storage means can be connected in parallel during the working mode and during the charging mode each can be connected to a charging means. Thus, the energy storage means can be charged using a smaller current and individual charging units, whereas in the working mode a higher current can be provided.

The prosthesis can be an endoprosthesis, in particular a prosthesis for extremities such as a finger, hand, forearm, elbow, arm and/or shoulder prosthesis. The prosthesis can also be a toe, foot, ankle, lower leg, knee, leg and/or hip prosthesis. In particular the prosthesis can be a prosthesis which is operated with an energy storage means whose output voltage is higher than the input voltage applied to the charging device, for example to a plug on the prosthesis. For example a prosthesis which is operated with a voltage supplied by a USB connection.

In this context prostheses include orthoses which do not replace the body part concerned, but rather, support it in its stabilization and movement.

The invention also includes a method for charging at least a first and a second energy storage means of a prosthesis, such that during the working mode the first energy storage means and the second storage means are connected in series, wherein

the first energy storage means and the second energy storage means are electrically separated from the consumer,

the series connection is removed,

the first energy storage means and/or the second energy storage means is/are electrically connected to a charging device.

In the charging mode the first energy storage means can be connected in parallel with the second energy storage means and/or connected electrically to the consumer separately.

In the charging mode the first and/or the second energy storage means can be separated from the consumer. In the working mode the first and/or the second energy storage means can be electrically separated from the charging device.

### BRIEF DESCRIPTION OF THE DRAWINGS

Below, the invention is explained in greater detail with reference to example embodiments and with the help of drawings. These explanations do not limit the protective scope, which is defined solely by the claims.

The drawings show:

FIG. 1: A hand prosthesis with an energy supply system,

FIG. 2: A second design of an energy supply system,

FIG. 3: A schematic representation of a forearm and hand prosthesis.

### DETAILED DESCRIPTION OF THE PREFERRED EMBODIMENTS

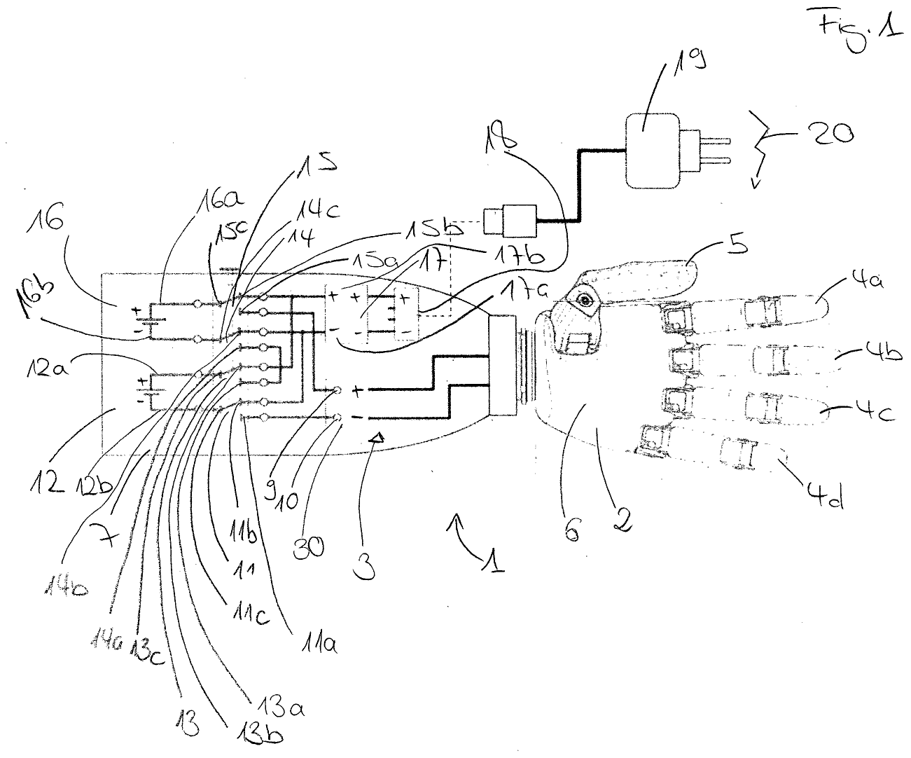

FIG. 1 shows a hand prosthesis 1 comprising a prosthesis body 2 and an energy supply system 3. The prosthesis body 2 comprises four finger elements 4a, 4b, 4c and 4d, a thumb element 5, and a hand body element 6. The finger elements 4a, 4b. 4c, 4d and the thumb element 5 are in each case connected pivotably to the hand body element 6. The finger elements 4a, 4b, 4c, 4d and the thumb element 5 can be pivoted by one or more electric motors. For this, the electric motors can be supplied by the energy supply system 3 with energy, in particular electrical energy.

The prosthesis body 2 further comprises a prosthesis shaft 7, wherein the prosthesis shaft 7 is rotatably connected to the hand body element 6 by way of a wrist element 8.

The energy supply system 3 can be arranged at least partially in the prosthesis shaft 7. Alternatively, the energy supply system 3 can also be arranged at least partially in the prosthesis body 2. The energy supply system 3 comprises at least two outputs 9 and 10, wherein one output 9 is connected to the electrically positive input of the consumers, such as the electric motors and/or the control electronic system 30, and the second output 10 is connected to the negative input of the consumers.

In the working mode, the first output 9 can then be connected via a positive pole 16a to at least a second accumulator 16. In the working mode the second output 10 can be connected to at least a negative pole 12b of the first accumulator 12.

The second output 10 is connected to a first input 11a of a first switch 11. In addition to the first input 11a, the first switch 11 has a second input 11b and an output 11c. The output 11c of the first switch 11 can optionally be connected to the first input 11a or to the second input 11b. The output 11c of the first switch is connected to the negative pole of the first accumulator 12.

Besides the switch 11, the energy supply system 3 can comprise a second switch 13, a third switch 14 and a fourth switch 15. The second switch 13 has a first input 13a, a second input 13b and an output 13c. The third switch 14 has a first input 14a, a second input 14b and an output 14c. The fourth switch 15 has a first input 15a, a second input 15b and an output 15c.

The output 11c of the first switch 11 can optionally be connected to the first input 11a of the first switch 11 or to the second input 11b of the first switch. The output 13c of the second switch 13 can optionally be connected to the first input 13a of the second switch 13 or to the second input 13b of the second switch 13. The output 14c of the third switch 14 can optionally be connected to the first input 14a of the third switch 14 or to the second input 14b of the third switch 14. The output 15c of the fourth switch 15 can optionally be connected to the first input 15a of the fourth switch 15 or to the second input 15b of the fourth switch 15.

The output 13c of the second switch 13 is connected to the positive pole 12a of the first accumulator 12, the output 14c of the third switch 14 is connected to the negative pole 16b of a second accumulator 16 and the output 15c of the fourth switch 15 is connected to the positive pole 16a of the second accumulator 16.

The second input 11b of the first switch is connected to the second input 14b of the third switch 14. The first input 13a of the second switch 13 is connected to the first input 14a of the third switch 14. The second input 13b of the second switch 13 is connected to the second input 15b of the fourth switch 15. The second input 14b of the third switch 14 is connected to the negative pole 17a of a charging unit 17. The charging unit is connected to a USB connector 18, which can be connected by way of a transformer 19 to a power supply system 20. The USB connector 18 can also be connected to another device which functions as a charging station or to a mobile energy source, such as a power-pack—a mobile accumulator charging unit.

The second input 15b of the fourth switch 15 is connected to the positive pole 17b of the charging unit 17. The positive output 9 is connected to the first input 15a of the fourth switch 15.

If the accumulators are to be charged, the output 11c of the first switch 11 is connected to the second input 11b of the first switch 11, the output 13c of the second switch 13 is connected to the second input 13b of the second switch 13, the output 14c of the third switch 14 is connected to the second input 14b of the third switch 14 and the output 15c of the fourth switch 15 is connected to the second input 15b of the fourth switch 15.

In this case the positive output 17b of the charging unit 17 is electrically connected to the positive pole 16a of the second accumulator 16 and to the positive pole 12a of the first accumulator 12, and the negative output 17a of the charging unit is connected to the negative pole 16b of the second accumulator 16 and to the negative pole 12b of the first accumulator 12. Thus, the positive output 9 and the negative output 10 are connected neither to the accumulators 12 and 16 nor to the charging unit 17.

If the consumers, such as the electric motors and the control electronics, are to be supplied with energy by the accumulators 12, 16, then the output 11c of the first switch 11 is connected to the first input 11a of the first switch 11, the output 13c of the second switch 13 is connected to the first input 13a of the second switch 13, the output 14c of the third switch 14 is connected to the first input 14a of the third switch 14 and the output 15c of the fourth switch 15 is connected to the first input 15a of the fourth switch 15. In this case, the positive output 9 is connected to the positive pole 16a of the second accumulator 16 and the negative output 10 is connected to the negative pole 12b of the first accumulator 12. The positive pole 12a of the first accumulator 12 is connected to the negative pole 16b of the second accumulator. The charging unit 17 is then not electrically connected to the accumulators 12 and 16.

The switches 11, 13, 14 and 15 can be individual mechanical, electric, electronic, magnetic or other switches, or they may form a switch combination which can be actuated as a whole. There can also be a relay.

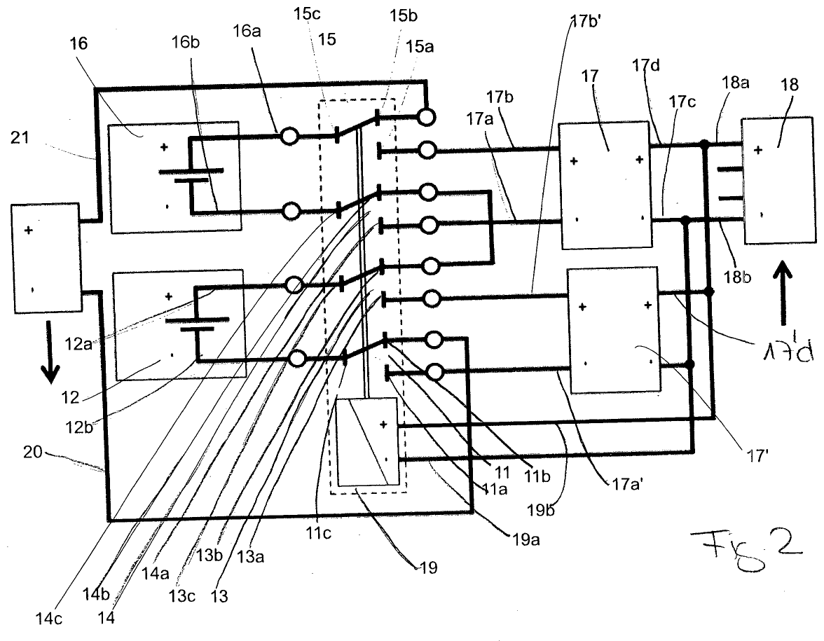

FIG. 2 shows an energy supply system 3 with a first accumulator 12 and a second accumulator 16. The first accumulator 12 is connected with its positive input 12a to the output 13c of the switch device 13. The negative input 12b of the first accumulator 12 is connected to the output 11c of the switch device 11. The positive input 16a of the second accumulator 16 is connected to the output 15c of the switch device 15. The negative input 16b of the second accumulator is connected to the output 14c of the switch device 14.

In a working mode in which the accumulators 12, 16 are electrically connected to one or more consumers in order to supply the consumers with electrical energy, the output 11c of the first switch device is connected to the second input 11b of the switch device 11, the output 13c is connected to the second input 13b, the output 14c is connected to the second input 14b and the output 15c is connected to the second input 15b. The second input 11b is connected to the negative pole 22 of an electrical connection to a consumer. The second input 13b is connected to the second input 14b. The second input 15b is connected to the positive pole 21 of an electrical connection to the consumer.

In a charging mode the output 11c is connected to the first input 11a, the output 13c is connected to the first input 13a, the output 14c is connected to the first input 14a and the output 15c is connected to the first input 15a. In this case the first input 11a is connected to a negative output 17a′ of a charging device 17′, the first input 13a is connected to a positive output 17b′ of the charging device 17′, the first input 14a is connected to a negative output 17a of a charging device 17 and the first input 15a is connected to a positive output 17b of the charging device 17.

A positive input 17d of the charging device 17 is connected to a positive input 17d′ of the charging device 17′ and to a positive output 18a of a low-voltage current supply. 18, such as a current supply according to the USB standard.

The low-voltage current supply can also be a USB connector.

A negative input 17c of the charging device 17 is connected to a negative input 17c′ of the charging device 17′ and to a negative output 18b of the low-voltage current supply 18.

The two outputs 18a, 18b of the low-voltage current supply 18 are in this case connected to two control inputs 19a and 19b of a relay 19, so that when a particular voltage is applied to the control inputs the relay switches and the output 11c is connected to the first input 11a, the output 13c is connected to the first input 13a, the output 14c is connected to the first input 14a and the output 15c is connected to the first input 15a.

If no voltage is applied at the control inputs, or a voltage whose value is below a specific threshold, then the output 11c is connected to the second input 11b, the output 13c is connected to the second input 13b, the output 14c is connected to the second input 14b and the output 15c is connected to the second input 15b.

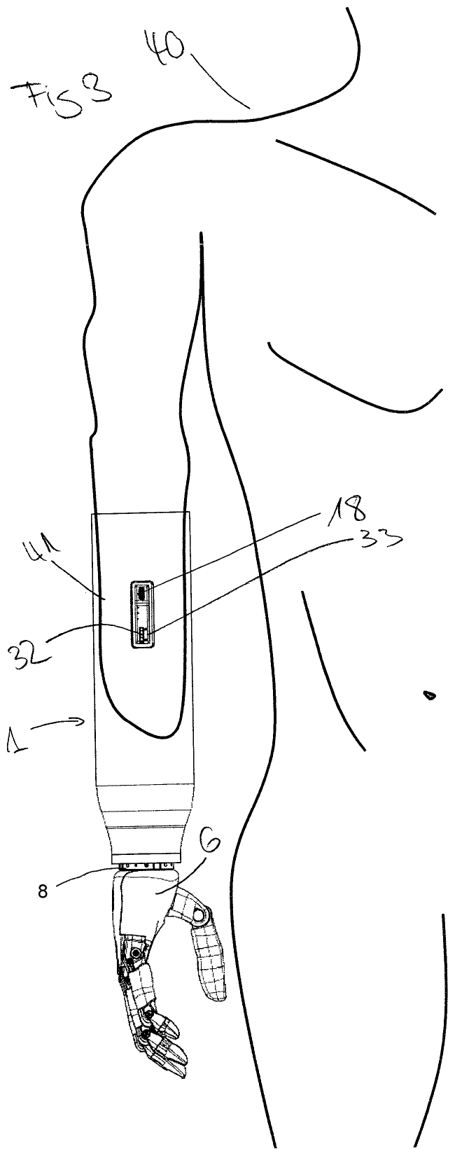

FIG. 3 shows a schematic illustration of a user 40 of a prosthesis 1. An arm stump 41 of the user 40 is in this case enclosed at least laterally by the prosthesis shaft 7 so that the prosthesis 1 is held by the arm stump 41. On the side of the prosthesis shaft 7 facing away from the arm stump 41 the hand body element 6 is connected to the prosthesis shaft 7 by a wrist element 8. The finger elements 4a, 4b, 4c, 4d and the thumb element 5 are arranged on the hand body element 6. On the outside of the prosthesis shaft a connection element such as a USB connector 18 can be arranged. Alongside the connection element a signal device 32 can also be arranged, which indicates whether the energy supply system is in the charging mode or the working mode. In addition a control element 33 can be arranged there, by means of which the respective mode of the energy supply system can be determined.

### INDEXES

1 Hand prosthesis
2 Prosthesis body
3 Energy supply system
4a Finger element
4b Finger element
4c Finger element
5 Thumb element
6 Hand body element
7 Prosthesis shaft
8 Wrist element
9 Output
10 Output
11 First switch
11a First input of the first switch
11b Second input of the first switch
11c Output of the first switch
12 First accumulator
12a Positive pole of the first accumulator
12b Negative pole of the first accumulator
13 Second switch
13a First input of the second switch
13b Second input of the second switch
13c Output of the second switch
14 Third switch
14a First input of the third switch
14b Second input of the third switch
14c Output of the third switch
15 Fourth switch
15a First input of the fourth switch
15b Second input of the fourth switch
15c Output of the fourth switch
16 Second accumulator
16a Positive pole of the second accumulator
16b Negative pole of the second accumulator
17 First charging device
17a Negative output of the first charging device
17b Positive output of the first charging device
17c Negative input of the first charging device
17d Positive input of the first charging device
17′ Second charging device
17a′ Negative output of the second charging device
17b′ Positive output of the second charging device
17c′ Negative input of the second charging device
17d′ Positive input of the second charging device
18 Low-voltage current supply
18a Output of the low-voltage current supply
18b Output of the low-voltage current supply
19 Relay
19a Control line of the relay
19b Control line of the relay
40 User
41 Arm stump

1-15. (canceled)

16. An energy supply system (3) for a prosthesis (1) comprising:
at least first and second energy storage devices (12, 16) for storing electrical energy,
wherein in a charging mode for charging at least the first energy storage device (12), the first energy storage device (12) is electrically connected, in a first connecting manner, to the second energy storage device (16), and, in a working mode for delivering electrical energy to a consumer, the first energy storage device (12) is electrically connected, in a second connecting manner, to the second energy storage device (16), and the first connecting manner is different from the second connecting manner.

17. The energy supply system (3) according to claim 16, wherein the first energy storage device (12), in the working mode, is connected in series with the second energy storage device (16).

18. The energy supply system (3) according to claim 16, wherein the first energy storage device (12), in the charging mode, is connected in parallel with the second energy storage device (16).

19. The energy supply system (3) according to claim 16, wherein the first energy storage device (12), in the charging mode, is electrically separated from the second energy storage device (16).

20. The energy supply system (3) according to claim 16, wherein at least one of the first and the second energy storage devices (12, 16) is an accumulator.

21. The energy supply system (3) according to claim 16, wherein at least one of the first and the second energy storage devices (12, 16) is a lithium-ion accumulator.

22. The energy supply system (3) according to claim 16, wherein the energy supply system (3) comprises a connection device according to a USB Standard.

23. The energy supply system (3) according to claim 16, wherein the energy supply system comprises at least the first energy storage device (12), the second energy storage device (16) and a third energy storage device, and, in the charging mode, a majority of the at least first, the second and the third energy storage devices (12, 16) are respectively connected in parallel with one of the first, the second and the third energy storage devices, and, in the working mode, the majority of the at least first, the second and the third energy storage devices (12, 16) are respectively connected in series with one of the first, the second and the third energy storage devices.

24. The energy supply system (3) according to claim 16, wherein the energy supply system comprises a manual switch for switching between the charging mode and the working mode.

25. The energy supply system (3) according to claim 16, wherein the energy supply system (3) comprises a detection device for detecting whether a charging device should be connected to the energy supply system.

26. The energy supply system (3) according to claim 16, wherein the first and the second energy storage devices, during the working mode, are connected in parallel and the first and the second energy storage devices, during the charging mode, are each connected to a charging device.

27. A prosthesis (1) with an energy supply system having at least first and second energy storage devices (12, 16) for storing electrical energy,
wherein in a charging mode for charging at least the first energy storage device (12), the first energy storage device (12) is electrically connected, in a first connecting manner, to the second energy storage device (16), and, in a working mode for delivering electrical energy to a consumer, the first energy storage device (12) is electrically connected, in a second connecting manner, to the second energy storage device (16), and the first connecting manner is different from the second connecting manner, and the prosthesis is an exoprosthesis.

28. The prosthesis (1) according to claim 27, wherein the prosthesis (1) is one of a prosthesis of an upper extremity, a finger prosthesis, a hand prosthesis, and an arm prosthesis.

29. A method for charging at least first and second energy storage devices (12, 16) of a prosthesis (1) starting from a working mode in which the first energy storage device (12) is connected in series with the second energy storage device (16), the method comprising:
electrically separating the first energy storage device (12) and the second energy storage device (16) from an energy consumer;
removing the series connection; and
electrically connecting, in a charging mode, at least one of the first energy storage device (12) and the second energy storage device (16) to a charging device (17, 17′) to initiate a charging mode.

30. The charging method according to claim 29, further comprising connecting the first energy storage device (12), in the charging mode, in parallel with the second energy storage device (16).
